(19) United States
(12) Patent Application Publication
(10) Pub. No.: US 2023/0015833 A1
(43) Pub. Date: Jan. 19, 2023

(54) Apparatus and method for personalized hormonal diagnostics and therapy

(71) Applicants: Natalia Shtraizent, Naperville, NY (US); Henry Grage, Johns Creek, GA (US); Falk Fish, Tel Aviv (IL)

(72) Inventors: Natalia Shtraizent, Naperville, NY (US); Henry Grage, Johns Creek, GA (US); Falk Fish, Tel Aviv (IL)

(21) Appl. No.: 17/857,096

(22) Filed: Jul. 4, 2022

Related U.S. Application Data
(60) Provisional application No. 63/218,695, filed on Jul. 6, 2021.

(51) Int. Cl. G01N 33/76; G01N 33/74; G16H 70/60; G16H 10/60

(52) U.S. Cl. CPC G01N 33/76; G01N 33/743; G16H 70/60; G16H 10/60

(57) ABSTRACT

A method and system for measuring hormone levels in human samples. Either actual levels can be measured, or hormone related parameters can be measured with computation extrapolating the parameters to the actual hormone level. A patient hormone level can be compared against a population database based on age, gender and other factors to determine a score for a particular hormone. A dosage of the hormone being given a patient can be automatically adjusted based on the score with optional approval of the adjustment made by a health professional. This is particularly useful in hormone replacement therapy and in-vitro fertilization.

This application is related to, and claims priority from, U.S. Provisional Patent Application No. 63/218,695 Filed Jul. 6, 2021. Application 63/218,695 is hereby incorporated by reference in its entirety.

### BACKGROUND

### Field of the Invention

The present invention is of an apparatus, system and method for personalized hormonal health, including without limitation, for diagnostic, prognostic and therapeutic applications, and in particular to such an apparatus, system and method for determining precision diagnostic for optimal individual hormonal level and personalized hormonal therapy, as well as for collecting data points related to the above applications for the purpose of personalized and population wide data analysis, by means of information technologies.

### Description of the Problem Solved

Hormonal Replacement Therapy (HRT) uses hormonal supplements to balance the hormonal levels of the healthy body. HRT is widely used to improve the outcomes of assisted reproduction. Frequently, hormonal supplementation is associated with negative side effects and increased health risk arising due to the variability in response to therapy among patients. For that reason, it is very important to first, determining the perfect dose for restoring the optimal balance and then to create a new, personalized approach, focused on tailoring the treatment to a patient. In addition to people undergoing fertility treatment, people with chronic conditions, such as depression, diabetes, fatigue, heart disease, insomnia, migraines/headaches, obesity, osteoporosis and familial predisposition to cancer prevention can benefit from better managing hormone misbalance. The method of the present invention offers a personalized medicine approach to improve healthcare management, clinical outcomes and the quality of life, for people with health conditions that require (or can benefit from) HRT.

Assisted reproductive technologies, including assisted fertilization and in vitro fertilization (IVF), rely on the evaluation of hormonal levels during different phases of menstrual cycle. In many clinics the standard clinical protocols dictate hormonal supplementation to override natural hormonal production and control the timing of ovulation, regardless of whether the woman has or does not have normal hormonal levels. An additional shortcoming in IVF is that, even when the hormonal replacement therapy is justified, the amount of hormones prescribed OB/GYNs and Endocrinologists is typically the same for all patients without considering their personal parameters, such as age, body mass index (BMI), or their current baseline hormonal levels. Non-personalized treatment usually leads to adding more drugs to treat the side effects (anti-depressants or sleep aid) creating a cocktail of medications that can cause even more imbalance and increase health risks.

The outcomes of this general protocol therapy are usually poor healthcare outcomes (due to extended trial and error period) and a heavy psychological burden of incompetence and inability to recover from the unhealthy state. With delayed child bearing age, the risk associated with hormonal replacement therapy goes up.

In the current practice, during each cycle of IVF. the patient needs to come for blood collection at least 5 times at a specific time that fits the clinic schedule, but not the natural personal timing. In addition, blood samples are sent out to an external lab, and the results are available only several hours later or sometime on the next day. Patients need a more accessible process, and more accurate timing of hormonal measurements. Using a personalized medicine approach, the doctor can stabilize unbalanced hormonal levels or can achieve the exact level needed during reproduction treatment with minimal side effects or health risks. Personalized HTR will include hormone testing, a personalize hormone therapy program that will include using specific algorithm based on individual parameters, such as age BMI hormonal levels goals, and by learning about the specific uptake and response of each patient. The goal is to insure maintaining optimum balance and hopefully assure that each patient will be able to balance his or her own hormone levels going forward.

### SUMMARY OF THE INVENTION

The present invention overcomes these limitations of the prior art by providing an apparatus, system and method for personalized precision hormonal therapy.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computing device”, a “computer”, or “mobile device”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, or a PDA (personal digital assistant). Any two or more of such devices in communication with each other may optionally comprise a “network” or a “computer network”.

### DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 shows a schematic block diagram of a non-limiting, exemplary embodiment of a device for measuring hormone levels. FIG. 1. Patient (1) provides a blood specimen (2) which is inserted into a single or multiple test device (3) which detect and measure multiple analytes in the said blood specimens. The results are detected and shown by suitable reader(s) (4), which are then telecommunicated (5) to central processing and storage unit (6), where the results are analyzed by dedicated software and human specialists, who decide and provide suitable medications (7). Later, the patient (1) is tested again by providing a fresh specimen (2) in order to validate the effect of treatment and decide how to continue.

FIGS. 2A and 2B show exemplary, non-limiting systems for personalized, precision hormone therapy for a subject according to at least some embodiments;

FIG. 3 shows an exemplary, non-limiting method for determining hormone levels in a subject according to at least some embodiments;

FIGS. 4A and 4B show exemplary, non-limiting methods for training and implementing a machine learning algorithm for determining hormone levels in a subject according to at least some embodiments;

FIG. 5 shows an exemplary, non-limiting method for constructing a personalized, precision hormone therapy for a subject according to at least some embodiments; and

FIG. 6 shows an exemplary, non-limiting method for training a new machine learning model, optionally with transfer learning, according to correlations of frequent measurements of hormones in the blood of subjects with therapeutic outcomes.

### DESCRIPTION OF PREFERRED EMBODIMENTS

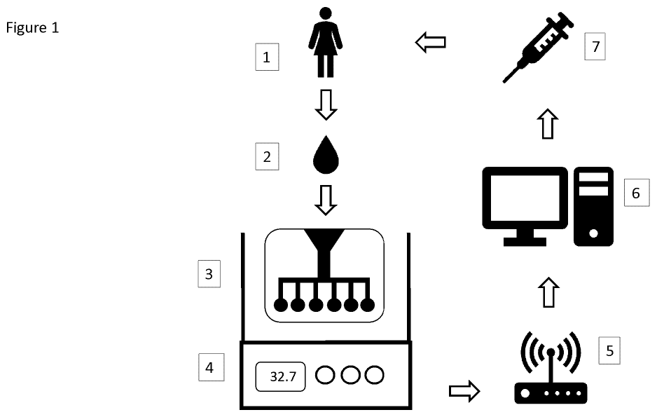

With regard to the apparatus of FIG. 1, optionally the following types of hormones are measured in the following ways designed for measuring hormones in peripheral blood. The methodologies described below are examples only, as various other methodologies could optionally be implemented.

Hormones to Be Tested and Ranges:

ESTRADIOL (E-2) pg/ml

Follicular Phase 0-1.13
Periovulatory (+/−3 days) 34.0-400
Luteal Phase 6.0-24
Postmenopausal 0.0-1.0
Range 1-1000 pg/ml. Error 50 pg/ml

Methodology:

Immunoassay (IA)

LH IU/L (1 IU=0.0465 ug)
Follicular Phase 0-1.13
Midcycle Phase 0.95-21.0
Luteal Phase (Day 7 to 8) 6.0-24
Postmenopausal 0.0-1.0
Range 1-100 IU/L (0-4.656 ug). Error 5 IU/L

Methodology:

Electrochemiluminescence immunoassay (ECLIA)

PROGESTERONE ng/ml
Follicular Phase 0-1.13
Midcycle Phase 0.95-21.0
Luteal Phase (Day 7 to 8) 6.0-24
Postmenopausal 0.0-1.0
Range 1-100 ng/ml. Error 1-2 ng/ml

Methodology:

ELISA

BETA-HCG mIU/ml(1 IU=0.06 ug)
2-4 weeks 39.1-8388
5-6 weeks 861-88,769
6-8 weeks 8,636-218,085
8-10 weeks 18,700-244,46
10-12 weeks 23,143-181,8
13-27 weeks 6,303-97,171
28-40 weeks 4,360-74,883
Range: Set one: 5-10,000 mIU/ml (0.3 ug-600 ug/L). Set One: Error 5 mIU/ml
   Set two: 10,000-300,000 mIU/ml (600 ug-18,000 ug/L).
   Set Two :Error 5,000 mIU/ml

Methodology:

Most tests employ a monoclonal antibody, which is specific to the β-subunit of hCG (β-hCG). This procedure is employed to ensure that tests do not make false positives by confusing hCG with LH and FSH.

The serum test, using 2-4 mL of venous blood, is typically a chemiluminescent or fluorimetric immunoassay that can detect βhCG levels as low as 5 mIU/ml and allows quantification of the βhCG concentration.

TESTOSTERONE nanograms per deciliter (ng/dL)
7 ng/dL-2,000 ng/dL
Range 7-2,000 ng/dL. Error 5 ng/dL

Methodology:

Immunoassay (electrochemiluminescent assay, ECLIA).
Follicle-stimulating hormone (FSH) (1 IU=113.8 ug)
Range 1-100 mIU/ml (113.8 ug/L-11,380 ug/L). Error: 100 ug/L

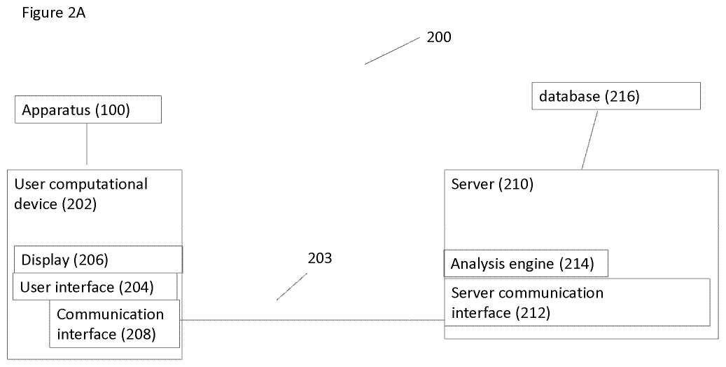

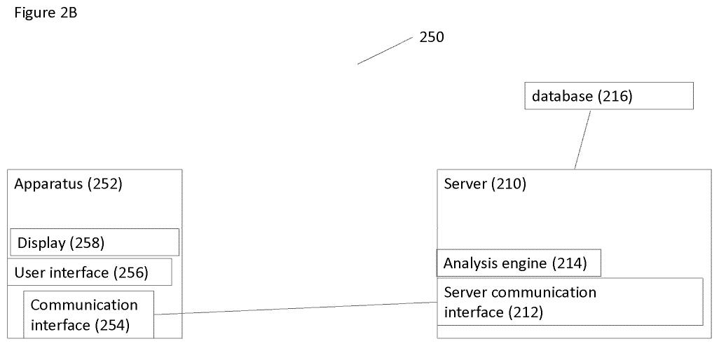

FIGS. 2A and 2B show non-limiting exemplary implementations of systems according to at least some embodiments of the present invention. FIG. 2A shows a first embodiment of a system 200 featuring apparatus 100 from FIG. 1, which may be implemented according to any suitable implementation for such an apparatus. Apparatus 100 determines at least hormone related parameters from measurements taken from the blood of the subject as previously described.

Apparatus 100 is in communication with a user computational device 202. User computational device 202 may optionally be a mobile phone, a smart phone, cellular telephone, desktop computer, laptop computer, or any other type of computational device. User computational device 202 is in communication with a server 210, preferably through a computer network such as network 203, as shown.

Server 210 preferably features an analysis engine 214 and a server communication interface 212 for communicating with a communication interface 208 on user computational device 202. Apparatus 100 passes the hormone related parameters to user computational device 202, whether through a hardwired connection, a portable cord, or some type of wireless connection, including but not limited to cellular, WiFi, Bluetooth and the like.

Display 206 optionally then shows one or more parameters related to the status of the user's, or in this case the subject's, blood hormone levels. User interface 204 enables the user to control user computational device 202 and, for example, to obtain then this information from apparatus 100. This information, such as the hormone parameter levels, may then optionally be analyzed on user computational device 202, but preferably this information is transmitted through communication interface 208 to server communication interface 212.

The information is then preferably analyzed on server 210 by an analysis engine 214. Analysis engine 214 may optionally include a machine learning algorithm or some other type of deep learning or artificial intelligence algorithm and the like. Optionally, analysis engine 214 is able to determine information such as the levels of hormone or hormones in the blood of the user, and/or whether adjustment should be made to the therapy and/or whether the user is meeting one or more therapeutic outcomes.

Optionally, analysis engine 214 takes information from database 216 in order to make these determinations. Information in database 216 may, for example, include but is not limited to population information on hormone levels, information regarding the correlation between hormone levels in the blood and the measured parameters by the device, correlations between hormone levels and therapeutic outcomes, optionally according to various characteristics of the user including but not limited to BMI, age, desired therapeutic outcome and the like, and also previous medical history.

Optionally then, such information is passed back from analysis engine 214 to user computational device 202 and may, for example, be displayed through display 206 and may also allow the user to interact with it through user interface 204, for example, to contact the user's doctor or other medical personnel in case there are questions.

FIG. 2B shows another implementation, in this case in a system 250. Preferably the components on the server side have not changed. However, in this case an apparatus 252 preferably measures the hormone related parameters in the blood as previously described, but now also includes communication interface 254, a user interface 256, and a display 258. These additional components enable all or nearly all of the analysis and communication to occur through apparatus 252.

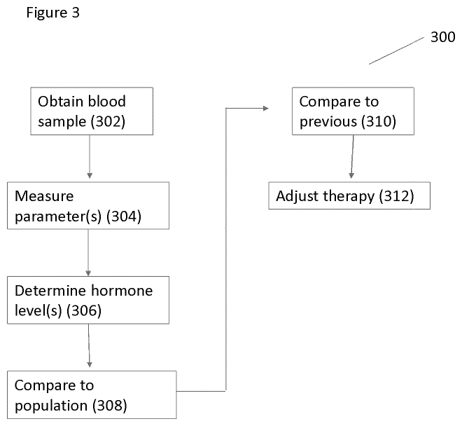

FIG. 3 shows a non-limiting exemplary method 300 for determining whether therapy needs to be adjusted according to one or more levels of hormones in the blood of the user. In step 302, a blood sample of the user is obtained, for example with the previously described apparatus. Next, one or more hormone related parameters are measured in step 304. As previously described, hormone related parameters include parameters that are measured by the apparatus which are indicative of a level of hormone, but which may not actually include an absolute determination of the hormone levels. Instead, preferably the hormone levels are calculated in step 306 from these measured parameters.

A hormone parameter may be the level of a non-hormone that directly relates to the hormone level; the level of a different hormone from which the level of the target hormone can be determined; the ratio of two hormones; the ratio of a hormone to a non-hormone; or the ratio of two non-hormones. Also combinations of the above examples of parameters may be used if they can be extrapolated to a particular hormone level. Non-hormones that may be used as parameters include enzymes, electrolytes and constituents of blood serum or whole blood. Examples of hormone parameters can be sex hormone-binding globulin (SHBG), Dehydroepiandrosterone (DHEA), 25-hydroxyvitamin D, parathyroid hormone (PTH), and insulin-like growth factor. Another possible example of a hormone parameter is the ratio of estradial to progesterone.

One advantage of measuring parameters rather than directly measuring hormone levels is that it is possible to make a simpler measurement and a simpler calculation on the apparatus. This information can then be correlated with hormone levels according to deeper algorithms which require more computational resources to operate, such as for example the previously described machine learning algorithms.

In step 302, these hormone levels may be compared to a population which has been selected for similarity to the user, for example according to BMI, age, and desired therapeutic outcome. This will help the medical personnel to determine whether the user fits within the previously determined population profile for a particular type of hormone therapy, or whether the user is perhaps an outlier. It may also provide a initial red flag or warning as to whether the therapy should be adjusted.

In step 310, the hormone levels are compared to previously obtained hormone levels. This is particularly important in the initial phases of the therapy when hormone levels are being increased as doses are administered over time and/or in situations where it's necessary for hormone levels to fluctuate, such as for example for IVF or other cyclical hormone related treatments and events. Optionally, in step 312 the therapy is adjusted according to either the population comparison or the previous hormone comparison or both.

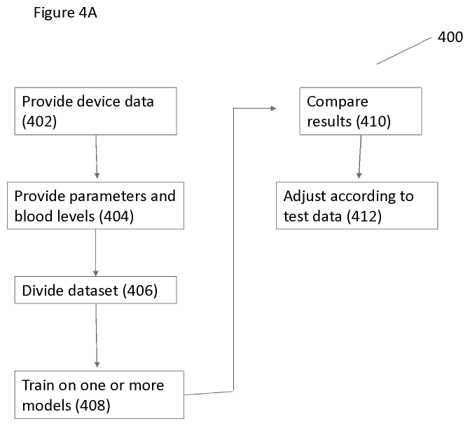

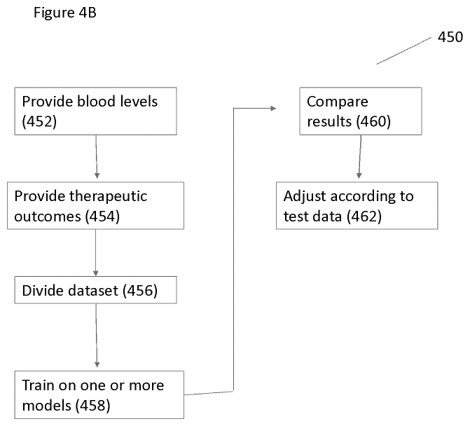

FIGS. 4A and 4B related to non-limiting exemplar methods for training machine learning algorithms, both to be able to determine hormone levels from the measured parameter by the apparatus and also to determine the personalized precise hormone therapy regimen for the particular user.

In a process 400, device data is provided in step 402. This is important because the device data itself relates to how the device is able to measure hormone levels. As previously noted, the device may not be capable of determining actual hormone levels but may only be able to provide one or more parameters that are hormone related. In any case, any device data which is provided preferably at least includes these hormone related parameters and may also include other types of calculations according to the ability of the device or apparatus to perform these calculations.

In step 404, the hormone related parameters and the correlated blood levels of hormone are also provided. This is important because the hormone related parameters may not themselves be directly related to hormone blood levels and instead a calculation may need to be performed to translate the hormone related parameters into hormone blood levels.

In step 406, preferably the data set is divided into a training and test data set. In step 408, the training data set is trained on one or more models. These are models for machine learning or deep learning. Preferably, training is performed on a plurality of models in order to determine which model performs best. It is possible that a particular model may perform differently, not only according to the type of hormone therapy being considered, but also according to the characteristics of the user and the amount and quality of data available for training.

In step 410, results are compared from a plurality models if in fact a plurality of models are trained. Then, optionally a feedback loop is performed in which the models are adjusted according to test data in step 412.

Optionally the models that are determined as described with regard to FIG. 4A involved supervised learning. Supervised learning will utilize known datasets of baseline datasets and target datasets to a) identify significant variables and generate prediction model of change from baseline to target datasets, as a consequence of medical intervention, and b) to define expected variance (y=ax²+bx+c), where x is an independent variable, representing the amount of the hormone required to cause the change from baseline to target value and y is the dependent value that changes corresponding to x, i.e. the predicted level of hormonal as a function of hormone dosage. Variance decomposition will be needed to identify contribution of additional variables to the unexplained variance of the dependable variant. Supervised learning will result in multi-parametric prediction model of normal, normal variance and abnormal variance.

Supervised learning utilizes known datasets (level/ranges of hormones A, B, C, D of multiple people, that do or don't require medical attention for hormonal replacement therapy) and target datasets baseline datasets (what is accepted a normal levels of hormones, i.e. previously established) to a) identify significant variables and generate prediction model of change from baseline to target datasets, as a consequence of medical intervention, and b) to define expected variance (y=a+bx+c), where x is an independent variable, representing the amount of the hormone required to cause the change from baseline to target value and y is the dependent value that changes corresponding to x, i.e. the predicted level of hormonal as a function of hormone dosage.

A non-limiting example is provided as follows. The normal range of hormone A is 10-15 U/mg at the beginning of the cycle and 100+/−15 U/mg at surge.
Woman 1 (25 y.o.) has 15 U/mg and 100 U/mg
Woman 2 (30 y.o.) has 20 U/mg and 75 U/mg
Woman 3 (35 y.o.) has 14 U/mg and 120 U/mg
Woman 4 (41 y.o.) has 12 U/mg and 100 U/mg
Woman 5 (25 y.o.) has 20 U/mg and 100 U/mg
20% of women have a fertility problem

1a. Machine learning will test different variables (for example, age) to establish the significant variable and create a prediction model change in hormonal levels as an indicator of fertility or infertility.

For example, are all women in the group age 20-25, who fall in the normal A ranges (10->100) fertile? What percent of fertile women falls in each age category. Is there a different normal range for different age group, and what is the correct age group division that makes the relationship significant?

2a) once significant normal ranges are established variance decompositions can be performed to identify additional factors that cause unexplainable variability, i.e., a woman is within the age range but her value falls outside of the predicted range, there could be an additional factor (for example, weight) that will define the variation within the age group.

Once the models or machine learning algorithms are ready to be used for determining blood hormone levels from hormone related parameters in the user, then training for a process 450, shown in FIG. 4B, is preferably performed. In step 452, blood levels of hormones are provided. These preferably also include correlations between the blood levels and also the hormone related parameters as previously described.

In step 454, therapeutic outcomes are provided according to which, whether a certain therapeutic outcome was achieved, whether side effects were obtained, whether problems occurred during the therapy and had to be adjusted or dropped, and any other medical outcome which is related to the effects of hormone therapy.

Again, in step 456 the data set is divided. Training is performed on one or more models in step 458, in this case to determine the correlation between hormone blood levels as measured through parameters measured by the device or apparatus and also the therapeutic outcomes, for example, as determined according to one or more population characteristics. In step 460 if a plurality of models are trained, then the results are compared and the results are then used to adjust the one or more test models according to the test data in 462.

The models established in the method of FIG. 4B are preferably prediction models, able to predict the response of at least a subset of users to hormone therapy. Prediction model—Mathematically established recommendation, taking in account significant variables (contributing parameters), for required adjustment in hormonal supplementation. Predicted outcome—The predicted level of hormone after recommended measure of hormonal supplementation.

Actual outcome—The level of hormone after hormonal supplementation. Predicted versus actual outcomes are preferably analyzed, as described in more detail below.

The training methods described in FIGS. 4A and 4B are designed to establish the type and the quantity of hormonal supplementation based on individual parameters (variables) to achieve the target value (normal range). The normal ranges will be predicted based on different variables using supervised learning and variance decomposition, to establish which variables need to be included in the prediction model for: Primary Generation: Primary use—diagnostic POCT test and device for improving the management of reproductive health. This diagnostic test will serve as 1) a tool for monitoring of the reproductive cycle (baseline and surge) during diagnosis of fertility/infertility, 2) a replacement of laboratory testing for at-home and in-clinic use (IVF and other applications); and 3) a utility for the development of personalized diagnostics and treatment in clinical trials. Secondary use (internal /investigational use)—A tool for the acquisition of clinical data linked to hormonal monitoring, including but not limited to: therapeutic drug monitoring following hormone administration, variations in hormones (Such as age, body weight and metabolic levels), and velocity of hormones (uptake and clearance—test every 8 h for two days), and monitoring of the secondary response, e.g. other hormones and other biomarkers. A device for big data acquisition, subsequent machine learning, and development of personalized reproductive medicine. Second generation 1) Personalized monitoring device enabling precision diagnostic and therapeutic application, 2) tool for reproductive telemedicine, 3) platform technology for hormonal monitoring and wellness. 4) Clinical trial tool.

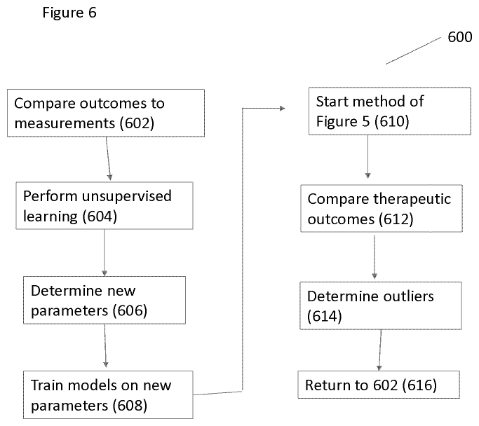

The process of establishment of the precision/personalized hormonal replacement will be done based on the outcomes of the intervention, through comparison of predicted outcomes to actual outcomes. The cases that don't fall within the predicted outcome will be called outliers and will become dataset 2 for the supervised model, for an extension of the method of FIG. 4B, as described with regard to FIG. 6. The continuous repeat of the supervised model will create the accurate model, needed for precision hormonal care.

A. Objectives of the Data Collection Using the Device:
1. Baseline level range among women of different ages and different BMI, ethnicity
2. The day of the peak
3. The peak level number
4. The difference between baseline and the peak
5. Other hormones at the same time

B. Predictions to Be Made Based on the Individual Patient Measurement and Monitoring and Analysis though AI-Guided Algorithm:
1. Optimal hormonal levels at baseline and fertility health
2. Timing of the ovulation and the fertility window.
3. Optimal HRT

C. Clinical Outcomes that Will Result from the Device and Algorithm:
1. If we can predict the peak of ovulation, we will know more accurate the reproductive window for successful fertilization.
2. If we can predict more accurately the hormonal levels (multiple hormones), we will predict optimal timing for embryo transfer—transfer or do not transfer, decrease pregnancy failure.
3. If we can predict optimal timing of embryo transfer, we will reduce the need for embryo reduction and high-risk pregnancy.
4. If we can detect personal optimal hormonal levels, we can improve quality of HRT and decrease adverse effects of HRT.

D. DATA to Be Collected

1. FSH blood levels on the beginning of cycle.

2. Estradiol blood levels during cycle (any time that has been measured for the specific cycle.

3. LH blood levels during cycle (any time that has been measured for the specific cycle including post LH hormonal induction).

4. Progesterone blood levels during cycle (any time that has been measured for the specific cycle including post insemination induction).

5. Anti-Mullerian Hormone (AMH) for assessment of ovarian reserve, for the purposes including, without limitation diagnostic of reproductive health, monitoring of the diminishing number of reproductive cells due to chemotherapy or development of AMH HRT, for clinical application, including but not limited to therapeutic monitoring and clinical intervention, such as AMH supplementation, exemplified by a method of inhibiting accelerated GV oocytes maturation due acute depletion of the M-1 as a result of chemotherapy, by the way of AMH supplementation. The level of AMH in the blood can help doctors estimate the number of follicles inside the ovaries, and therefore, the woman's egg count. A typical AMH level for a fertile woman is 1.0-4.0 ng/ml; under 1.0 ng/ml is considered low and indicative of a diminished ovarian reserve, also level of AMH can change during cancer therapy).

6. TSH blood levels if have in record.

7. Number of follicles and size.

8. Endometrium thickness.

9. Number of oocyte.

10. Weight and Height (BMI).

10. Medical precondition history information (polycystic ovary, chemotherapy premenopausal, and so forth).

11. Ovarian reserve (volume of ovaries based on ultrasound).

12. Information about hormonal treatment for each cycle. Name of drug, units number for each hormone and administration method.

12. Outcome of cycle.

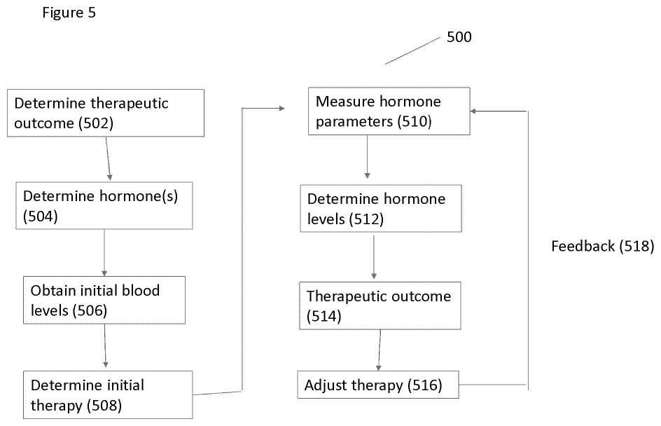

FIG. 5 relates to a method 400 for determining a particular therapeutic outcome and for adjusting hormone therapy as necessary for a user. This flow is preferably performed separately for each individual user. In step 502, the therapeutic outcome to be achieved is determined. This preferably includes not only hormone levels but also therapeutic effects, which may include for example the ability of a hormone to induce an increase or a decrease in another hormone level, an outcome such as pregnancy, and the like.

Next, in 504 one or more hormones which may be selected for such therapy are considered. Preferably, at least one hormone is then selected for initial therapy. In step 506, initial blood levels of the hormones are obtained as previously described. This may, of course, be by determining hormone related parameters and then translating these into initial blood levels of hormones. Determining the initial blood levels of hormones may be determined by a machine learning algorithm as previously described.

In step 508, initial therapy is determined which may also be performed by a machine learning algorithm. In step 510, hormone parameters are measured in the blood after the user has begun hormone therapy. In step 512, the actual hormone levels are determined as previously described, and in step 514 these are correlated with the current therapeutic outcome. In step 516, one or more machine learning algorithms may determine that it is necessary to adjust the therapy and this is provided as feedback in step 518. Optionally, this process is performed a plurality of times until the desired therapeutic outcome is achieved or until it is determined that the particular therapy is proving ineffective.

The process is optionally adjusted according to the type of hormone therapy. As a non-limiting example, in HRT ideally the dosage and frequency of administration should be adjusted to ensure that plasma hormone concentrations remain in the mid-normal range 1 week after administration and within the normal range until the next dose. Hormonal plasma levels should be measured 24-48 hours after administration.

The type of hormone therapy and/or the expected therapeutic outcome is not limiting for the invention as described herein. For example, and without limitation, hormone therapy could be performed for HRT (Hormone Replacement Therapy), IVF (in vitro fertilization) as a non-limiting of ART (assisted reproductive technology), male hormone (testosterone for performance), growth hormone treatment for children, gender reassignment therapy, hormone inhibitor treatment (for example for cancer treatment) and so forth.

Some of the conditions that benefit from balancing hormones include but are not limited to depression, diabetes, fatigue, heart disease, insomnia, migraines headaches, obesity, osteoporosis and cancer prevention/treatment.

The above process preferably reduces side effects, which can be significant under current medical practice as individuals react differently to different hormone regimens. Treatment with hormone agonists can cause life threatening side effects, due to overstimulation of the hormone system. On the other hand, giving inhibitors can cause side effects due to over treatment.

Various types of dosages can be used with the process of FIG. 5, including but not limited to pill, injection, deposition (for example for intramuscular injection), sticker or other topical administration, etc.

FIG. 6 shows an exemplary, non-limiting method for training a new machine learning model, optionally with transfer learning, according to correlations of frequent measurements of hormones in the blood of subjects with therapeutic outcomes. As detailed studies with such frequent measurements are not currently available. This method is preferably performed after frequent measurement data, with the apparatus as described herein, is available.

As shown in a process 600, therapeutic outcomes are compared to daily measurements in step 602. In this case, the process could involve sorting users into two groups: therapeutic outcomes were as expected, as guidance provided from the previously described models enabled therapeutic goals to be achieved; or therapeutic outcomes were not as expected, despite such guidance. The latter group of users can be described as “outliers”. Preferably additional medical data is available regarding such users, such as for example the presence of other medical conditions, to assist in training new models.

Unsupervised learning method will be applied to compare unknown datasets obtained from the biological sample testing to the target datasets, to identify outliers. Unknown data sets, comprised of outliers will be added into the supervised learning model, to refine significant variance decomposition and achieve significance in the subsets.

In step 604, unsupervised learning is preferably performed on the expanded dataset of information on outliers to determine which parameters are important. New parameters and information are then preferably selected in step 606. Machine learning models can be trained on the new data and information in step 608.

Once new models were available, the process could optionally move to the process described in FIG. 5, or any other suitable therapeutic process in which the new models are implemented to guide precision personalized hormone therapy, in 610.

Next therapeutic outcomes for the new models are again compared to the frequent daily measurements of hormone levels, to determine whether the new models again result in outlier users, in 612. Outliers are then determined in 614 and the process returns to 602 at step 616. To date the hormonal treatment protocol is general and limited. Obstetrics and gynecology physicians (OB/GYNs) and other specialists prescribe the same doses and the same drug (for example birth control pills) to all patients without considering their personal parameters, such as age, body mass index, or their current hormonal levels. Non-personalized treatment usually leads to adding more drugs to treat the side effects (antidepressants or sleep aid), creating a cocktail of medications that can cause even more imbalance and increase health risks. This generic protocol therapy usually leads to poor healthcare outcome low treatment compliance and increase in off-label prescribing. In addition, it poses a heavy psychological burden and inability to recover from the unhealthy state. With delayed childbearing age, the risk associated with inadequate hormonal treatments goes up.

All hormonal therapy patients are at risk for severe ovarian hyper-stimulation syndrome (OHS) that can lead to rapid weight gain—more than 2.2 pounds (1 kilogram) in 24 hours, severe abdominal pain, persistent nausea and vomiting, blood clots, decreased urination, shortness of breath, and tight or enlarged abdomen. Risk Factors that are known to increase risk of OHS include: Polycystic ovary syndrome, stimulation of a large number of follicles, age under 35, low body weight, high or steeply increasing level of estradiol (estrogen) before an HCG trigger shot, previous episodes of Ovarian hyper-stimulation syndrome (OHS).

Severe ovarian hyper-stimulation syndrome is an uncommon, but life-threatening condition, with one or more of the following symptoms: Fluid accumulation in the abdomen and sometimes the chest, electrolyte disturbances (sodium, potassium, others), blood clots in large vessels, usually in the legs, kidney failure, twisting of an ovary (ovarian torsion), rupture of a cyst in an ovary, which can lead to serious bleeding, difficulty breathing, miscarriage or pregnancy termination because of complications, and occasionally death (1%).

Other potential risks include increased levels of anxiety and depression, ectopic pregnancy, pre-eclampsia, placenta praevia, placental separation and increased risk of cesarean section (Ref 3-7).

For women undergoing hormonal Therapy as part of reproductive care, there is a need to optimize the regiment to decrease side effects of the therapy, decrease the risk of serious side effects and improve clinical outcomes. For women undergoing hormonal therapy during menopause to decrease the risk of osteoporosis and better manage menopause symptoms, there is a need to optimize the regimen to achieve improved symptom control. For women with signs of premature menopause, a completely personalized protocol may be needed to extend the fertility window.

The present invention provides a method to optimize the dose of the hormone(s) as follows:
1. Learn and to create new algorithms based on existing large data sets, and from the future machine learning techniques and new data. Perform data exploration to define the value parameters associated with personal hormonal levels and personal dynamic changes in hormonal levels; identify subgroups with common patterns which are potentially link to adverse effects, health risks and long term complications of current standard of care in hormonal therapy. The criteria will include: date of birth (DOB), Sex and gender, weight, height, and body mass index (BMI).
2. Perform validation of personal dosing by monitoring hormone absorption and clearance in the blood post consuming a specific known dose. Therapeutic drug monitoring (TDM). TDM will be done by several measurement during 48 hours post taking the known dose. Repeating the test every 6-8 hours will help to monitor drug/hormone elevation and/or reduction in the blood. Once the validation is completed, a personalized dose can be giving to achieve the goal level for the treatment. TMB is presently utilized for different type of drugs aimed at specific illnesses (diabetes, cardiovascular disease, kidney disease t thyroid disease, liver disease, and HIV) but is not used yet in hormonal treatment. TDM is commonly used to help physicians monitored maintain drug levels within a particular therapeutic window. The therapeutic window is the concentration range in which the drug exerts its clinical effect with minimal adverse effects for most patients and can prevent over dosing. TDM should be utilized for all Hormonal replacement therapies such as Estradiol (E2), Progesterone and Testosterone. Also, hormonal inhibitor therapy as a cancer treatment such as Aromatase inhibitors, selective estrogen receptor modulators and estrogen receptor antagonists
3. Monitoring hormone blood levels more frequently to improve the outcome of the treatment, for example, LH surge case use. An LH surge occurring over two days will have a peak level with “shoulders” (rising and falling) on either side of the peak. If the frequency of testing is too low, for example, two days or greater, it is possible to completely miss the peaks and the shoulders. If the frequency of testing one day, the possibilities are that only the peak or only the shoulders will be detected. Depending on the height of those shoulders, they may or may not be detectable above the patient's baseline level—the determination may be uncertain. If the frequency of testing is every twelve hours, there is a good chance of catching the peak. In this case, the two shoulders would be recognized even if they were close to the patient's baseline. In a case where the peak is missed, at least two near-peak shoulder levels would be detected. And, of course, if measurements were taken ever six hours (or more frequently), more detectable levels of the surge will be seen

In summary, the present invention relates to a method of human hormone monitoring and control that includes:
(a) taking a human blood or serum sample from a particular patient;
(b) measuring a hormone parameter from the sample;
(c) determining a level of a particular hormone from the hormone parameter;
(d) comparing the level of the particular hormone against a population database containing a sample space of a particular population to determine a score based on whether the hormone level falls in a normal region for said particular population;
(e) automatically adjusting dosage of said particular hormone to the particular patient based on the score.
(f) requiring a medical professional to allow or disallow said automatically adjusting in step (e) before the adjusting occurs.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of human hormone monitoring and control comprising:
(a) taking a human blood or serum sample from a particular patient;
(b) measuring a hormone parameter from said sample;
(c) determining a level of a particular hormone from the hormone parameter;
(d) comparing the level of the particular hormone against a population database containing a sample space of a particular population to determine a score based on whether the hormone level falls in a normal region for said particular population;
(e) automatically adjusting dosage of said particular hormone to the particular patient based on the score.

2. The method of claim 1, further comprising requiring a medical professional to allow or disallow said automatically adjusting in step (e) before said adjusting occurs.

3. The method of claim 1, further comprising communicating a patient identification (ID); a hormone ID; a population ID; and said score to a remote location.

4. The method of claim 1, wherein said at least one parameter is a ratio of two hormones.

5. The method of claim 1, wherein said at least one parameter is a level of a non-hormone.

6. The method of claim 1, wherein said particular population is female.

7. The method of claim 6, wherein said particular population is divided into age sub-groups, wherein there is a normal region for each age sub-group.

8. The method of claim 6, wherein said particular population is divided into subgroups that include at least one of: follicular phase, preiovulatory phase, midcycle phase, or luteal phase.

9. The method of claim 8, further comprising the subgroup postmenopausal.

10. The method of claim 1, wherein the particular hormone is estradiol or progesterone.

11. The method of claim 1, wherein the particular hormone is chosen from the group consisting of luteinizing hormone (LH), beta-hCG, follicle-stimulating hormone (FSH), and testosterone.

12. The method of claim 1, wherein said at least one hormone parameter is a ratio of estradiol to progesterone.
Luteinizing hormone (LH), beta-hCG, Follicle-stimulating hormone (FSH), testosterone.

13. The method of claim 1, wherein the hormone parameter is chosen from the group consisting of: sex hormone-binding globulin (SHBG), Dehydroepiandrosterone (DHEA), 25-hydroxyvitamin D, parathyroid hormone (PTH), and insulin-like growth factor.

14. A method of human hormone monitoring and control comprising:
(a) taking a sample from a particular patient;
(b) measuring a plurality of hormone parameters from said sample;
(c) determining a level of a particular hormone from the plurality of hormone parameters;
(d) comparing the level of the particular hormone against a population database containing a sample space of a particular population to determine a score based on whether the hormone level falls in a normal region for said particular population;
(e) automatically adjusting dosage of said particular hormone to the particular patient based on the score.

15. The method of claim 14, further comprising requiring a medical professional to allow or disallow said automatically adjusting in step (e) before said adjusting occurs.

16. The method of claim 14, wherein the particular hormone is chosen from the group consisting of luteinizing hormone (LH), beta-hCG, follicle-stimulating hormone (FSH), and testosterone.
